SEQUENCE LISTING (2 sequences)

SEQ ID NO: 1 - DNA, 24 bp, Artificial Sequence
  Description of Artificial Sequence: Synthetic polynucleotide
tttgggttgagtggagcctcggtc

SEQ ID NO: 2 - DNA, 24 bp, Hermetia illucens
tttcaacggtcgccaggctagggt

(19) United States
(12) Patent Application Publication
(10) Pub. No.: US 2023/0172175 A1
(43) Pub. Date: Jun. 8, 2023

(54) RECOMBINANT PROTEIN PRODUCTION IN INSECTS

(71) Applicant: Proteinea, Inc., San Francisco, CA (US)

(72) Inventors: Mahmoud Eljendy, Cairo (EG); Abdulaziz Elgammal, Cairo (EG); Ahmed Adel, Nasr City (EG); Rana Hashish, Nasr City (EG); Youssef Madbouly, Shorouk City (EG); Ahmed Marouf, Cairo (EG)

(21) Appl. No.: 17/911,930

(22) PCT Filed: Mar. 15, 2021
(86) PCT No.: PCT/US2021/022389

§ 371 Date: Sep. 15, 2022

Related U.S. Application Data
(60) Provisional application No. 62/989,725, filed on Mar. 15, 2020.

(51) Int. Cl. A01K 67/033; C12N 9/12; C12N 15/85

(52) U.S. Cl. CPC A01K 67/0339; C12N 9/1241; C12N 15/8509; A01K 2217/052; A01K 2217/203; A01K 2227/706; A01K 2267/01

(57) ABSTRACT

The present disclosure relates to the field of commercial scale production and processing of pharmaceutical liquid or solid compositions derived from insects, wherein the compositions include a purified recombinant protein, vaccine, antibody, peptide, or chemical. Systems and methods to produce the insects and a purified insect-derived recombinant protein, vaccine, antibody, peptide, insecticide, fungicide, or chemical within a bioreactor are also described.

### RELATED APPLICATIONS

This application claims the benefit of priority to U.S. Provisional Pat. Application Serial No. 62/989,725, filed Mar. 15, 2020, the contents of which are hereby incorporated herein by reference in their entirety.

### FIELD OF THE INVENTION

The present invention is in the field of recombinant biotechnology, in particular in the field of protein expression. The invention is directed to the efficient production of recombinant proteins in batch and/or continuous modes using insect larvae or pupae. In particular, those belonging to the group Hermetia illucens commonly known as black soldier fly, Gryllodes sigillatus commonly known as tropical house cricket, Gryllus assimilis commonly known as Jamaican field cricket, Acheta domesticus commonly known as house crickets, Tenebrio molitor commonly known as yellow mealworm, Tenebrio obscurus commonly known as dark mealworm, Alphitobius diaperinus commonly known as lesser mealworms. The invention generally relates to a method of expressing a protein of interest (POI) from a host organism by means of genetic transformation. Moreover, the present invention is also directed to the transformed insect itself comprising an exogenous nucleic acid.

### BACKGROUND

The advent of recombinant DNA technology is a major breakthrough that enabled the production of recombinant proteins in a variety of heterologous systems. Recombinant proteins have a wide range of applications in food, beverages, cosmetics and pharmaceutical industries (Puetz, J., et al. Processes 2019, 7, 476). Diverse platforms can be used for the production of recombinant proteins for industrial and therapeutic uses including bacteria, yeast, fungi, plant, mammalian, and insect cells. However, these different host systems have several limitations. Prokaryotic culture systems as bacteria can be used to produce small and short peptides, but cannot produce complex proteins that require folding and post-translational modifications such as glycosylation (Puetz, J,, et al. Processes 2019, 7, 476), The use of yeast can be advantageous to partly overcome this limitation and for the relatively simple downstream purification of the produced protein. However, the use of yeast is not suitable for the production of mammalian or proteases-sensitive proteins (Palomares, L., et al. Methods in Molecular Biology 2004,267). The use of animal cells such as mammalian and insect cells can overcome multiple limitations. In particular, Chinese Hamster Ovary cells (CHO) are the gold standard platform for the production of mammalian therapeutic proteins. Nevertheless, the high cost of the media for culturing animal cells is a challenging constraint in large-scale production (Puetz, J., et al. Processes 2019, 7, 476). The cost of production and the linear cost of bioreactor-based systems has been hindering the potential uses of protein-based products in different industries. In regard to this, the development of innovative platforms for rapid and cost-effective production of recombinant proteins at a large-scale is of paramount importance. Therefore, there is a need for alternative methods of producing recombinant proteins.

### SUMMARY OF THE INVENTION

The present invention discloses methods for commercial production of recombinant proteins in Hermetia illucens,Gryllodes sigillatus, Gryllus assimilis, Acheta domesticus, Tenebrio molitor, Tenebrio obscurus, Alphitobius diaperinus including generating copies of the gene coding for the recombinant protein of interest, followed by delivery of the gene of interest into the insect body by adopting different approaches to mediate its expression in the host. One object of the current invention is to provide a platform for generating recombinant proteins of commercial potential using insects, which is expected to have multiple advantages such as being cost-effective, having a rapid production cycle and high production yields. Thus, representing a promising alternative to the commonly used platforms.

In one aspect, disclosed herein is a transformed insect comprising at least one exogenous gene, wherein the insect is of a genus selected from the group consisting of Hermetia, Gryllodes, Gryllus, Acheta, Tenebrio, and Alphilobius. Numerous embodiments are further provided that can be applied to any aspect of the present invention described herein. For example, in some embodiments, the exogenous gene is comprised within somatic cells of the insect or the exogenous gene is comprised within the germline of the insect. In some embodiments, the exogenous gene is stably heritable. In some embodiments, the insect is a species genus selected from the group consisting of Hermetia illucens, Gryllodes sigillanis, Gryllus assimilis, Acheta domesticus, Tenebrio molitor, Tenebrio obscurus, or Alphitobius diaperinus, preferably the insect is Hermetia illucens. In some embodiments, the insect, is a mealworm selected from the group consisting of Tenebrio molitor, Tenebrio obscuras and Acheta domesticus. In some embodiments, the insect is an embryo, larva, pupa or adult. In some embodiments, the insect expresses a recombinant protein encoded by the at least one exogenous gene. In some embodiments, the insect expresses at least 2 mg, 5 mg, 10 mg, 15, mg, 20 mg, or 25 mg of the recombinant protein or at least 30 mg, 35 mg, 40 mg, 45 mg of the recombinant protein. In some embodiments, the recombinant protein is at least 10% 15%, 20%, 25%, 30%, 35%, 40%, 45%, 50%, 55%, 60%, 65%, or 70% of the total protein expressed by the insect. In some embodiments, the recombinant protein is selected from the group consisting of an antibody, an antigen, an antigen-binding molecule, an enzyme, a hormone, a component of a vaccine, and a virus-like particle. In some embodiments, the at least one exogenous gene is integrated into the insect genome by transposon-mediated integration. In some embodiments, the at least one exogenous gene is under the control of drosophila melanogaster hsp70 promoter.

In one aspect, disclosed herein is a reared insect derived from the transformed insect disclosed herein, wherein the reared insect comprises the at least one exogenous gene. Numerous embodiments are further provided that can be applied to any aspect of the present invention described herein. For example, in some embodiments, the reared insect expresses a recombinant protein encoded by the at least one exogenous gene. The reared insect may be removed by 1, 2, 3, 4 or 5 generations from the transformed insect or may be removed by greater than 5 generations from the transformed insect

In one aspect, disclosed herein is a pupa derived from the transformed insect, described herein or from the reared insect described herein. Numerous embodiments are further provided that can be applied to any aspect of the present invention described herein.

In one aspect, disclosed herein is a larva derived from the transformed insect disclosed herein or from the reared insect disclosed herein. Numerous embodiments are further provided that can be applied to any aspect of the present invention described herein.

In one aspect, disclosed herein is an egg derived from the transformed insect disclosed herein or from the reared insect disclosed herein. Numerous embodiments are further provided that can be applied to any aspect of the present invention described herein.

In one aspect, disclosed herein is an adult insect derived from the transformed insect disclosed herein or from the reared insect disclosed herein Numerous embodiments are further provided that can be applied to any aspect of the present invention described herein.

In one aspect, disclosed herein is a biomass produced from the transformed insect disclosed herein or from the reared insect disclosed herein. Numerous embodiments are further provided that can be applied to any aspect of the present invention described herein.

In one aspect, disclosed herein is recombinant protein isolated from the transformed insect disclosed herein or from the reared insect disclosed herein. Numerous embodiments are further provided that can be applied to any aspect of the present invention described herein. For example, in some embodiments, the protein is selected from the group consisting of an antibody, an antigen, an antigen-binding molecule, an enzyme, a hormone, a component of a vaccine, and a virus-like particle.

In one aspect, disclosed herein is a method for producing a protein comprising: a. transforming a first stage of an insect selected from the group consisting of Hermetia illucens, Gryllodes sigillatus, Gryllus assimilis, Acheta domesticus, Tenebrio molitor, Tenebrio obscurus, or Alphitobius diaperinus, wherein the transformation results in the integration of an expression cassette into the genome of the insect and wherein the expression cassette comprises a gene encoding a heterologous protein, wherein the heterologous gene is under the control of a heat shock promoter; b. heat-shocking the transformed insect at a second stage; and c. harvesting biomass comprising the protein from the transformed insect. Numerous embodiments are further provided that can be applied to any aspect of the present invention described herein. For example, in some embodiments, the first stage is an egg or an embryo. In some embodiments, the second stage is a larva, a pupa or an adult.

In one aspect, disclosed herein is a method for producing a protein comprising: a, transforming an insect selected from the group consisting of Hermetia illucens, Gryllodes sigillatus, Grylius assimilis, Acheta domesticus, Tenebrio molitor, Tenebrio obscurus, or Alpliitobius diaperinus, wherein the transformation results in the integration of an expression cassette into the germline of the insect and wherein the expression cassette comprises a gene encoding a heterologous protein, b. breeding the transformed insect to produce a subsequent generation insect; and c. harvesting biomass comprising the protein from the subsequent generation insect. Numerous embodiments are further provided that can be applied to any aspect of the present invention described herein. For example, in some embodiments, the method further comprises a step of mass rearing the subsequent generation insect, prior to the step of harvesting the biomass. In some embodiments, the transforming step is performed at the insect egg or embryo stage. In some embodiments, the biomass is harvested from larva, pupa or adult stages. In some embodiments, the heterologous gene is under the control of a heat shock promoter, and the method further comprises heat shocking the subsequent generation insect prior to harvesting the biomass. In some embodiments, the heat shock promoter is the drosophila melanogaster hsp70 promoter In some embodiments, the insect expresses at least 2 mg, 5 mg, 10 mg, 15, mg, 20 mg, or 25 mg of the heterologous protein or the insect expresses at least 30 mg, 35 mg, 40 mg, 45 mg of the heterologous protein. In some embodiments, the heterologous protein is at least 10% 15%, 20%, 25%, 30%, 35%, 40%, 45%, 50%, 55%, 60%, 65%, or 70% of the total protein expressed by the insect. In some embodiments, the method further comprises isolating the heterologous protein from the biomass. In some embodiments, the method further comprises enriching or partially purifying the heterologous protein from the biomass. In some embodiments, the transforming step comprises transposon-mediated integration of the expression cassette. In some embodiments, the transforming step further comprises providing a transposase enzyme active in the insect, and wherein the expression cassette is flanked at each end by a transposable element. In some embodiments, the transposase enzyme is provided as a coding region on a helper plasmid or a coding region encoded by a nucleic acid or as a transposase protein. In some embodiments, the insect is a Hermetia illucens. In some embodiments, the insect is a mealworm selected from the group consisting of Tenebrio molitor, Tenebrio obscurus and Acheta domesticus. In some embodiments, the transforming step comprises physical or chemical delivery of the expression cassette. In some embodiments, the transforming step comprises injection, DNA particle bombardment, electroporation, injection followed by electroporation, hydrodynamic, ultrasound, or magnetofection. In some embodiments, the heterologous protein is selected from the group consisting of an antibody, an antigen, an antigen-binding molecule, an enzyme, a hormone, a component of a vaccine, and a virus-like particle.

In one aspect, disclosed herein is a heterologous protein bio-manufactured by any one of the methods disclosed herein. Numerous embodiments are further provided that can be applied to any aspect of the present invention described herein. For example, in some embodiments, the expression vector expresses virus-like particles (VLP).

In one aspect, disclosed herein is a VLP bio-manufactured by any one of the methods disclosed herein. Numerous embodiments are further provided that can be applied to any aspect of the present invention described herein.

In one aspect, disclosed herein is a vaccine component bio-manufactured by any one of the methods disclosed herein. Numerous embodiments are further provided that can be applied to any aspect of the present invention described herein. For example, in some embodiments, the method further comprises isolating the heterologous protein from the whole body of the transformed larvae. In some embodiments, the method further comprises isolating the heterologous protein from the hemolymph, the fat body, or secretory glands of the transformed larvae.

In one aspect, disclosed herein is an isolated heterologous protein produced by any one of the methods of disclosed herein. Numerous embodiments are further provided that can be applied to any aspect of the present invention described herein.

In one aspect, disclosed herein is a method for producing transformed insect comprising transforming an insect egg or embryo of Hermetia illucens by providing a transposase enzyme active in the insect, and an expression cassette, wherein the expression cassette comprises a gene encoding a heterologous protein and wherein the expression cassette is flanked at each end by a transposable element, whereby the transformation results in the integration of the expression cassette into the genome of the insect. Numerous embodiments are further provided that can be applied to any aspect of the present invention described herein. For example, in some embodiments, the transposase enzyme is provided as a coding region on a helper plasmid or a coding region encoded by a nucleic acid or as a transposase protein. In some embodiments, the expression cassette is comprised within somatic cells of the insect. In some embodiments, the expression cassette is comprised within the germline of the insect. In some embodiments, the insect expresses at least 2 mg, 5 mg, 10 mg, 15, mg, 20 mg, or 25 mg of the heterologous protein or the insect expresses at least 30 mg, 35 mg, 40 mg, 45 mg of the heterologous protein. In some embodiments, the heterologous protein is at least 10% 15%, 20%, 25%, 30%, 35%, 40%, 45%, 50%, 55%, 60%, 65%, or 70% of the total protein expressed by the insect. In some embodiments, the method further comprising harvesting the heterologous protein from the insect. In some embodiments, the method further comprises: a. breeding the transformed insect to produce a subsequent generation insect; and b. harvesting biomass comprising the heterologous protein from the subsequent generation insect.

In one aspect, disclosed herein is a transformed insect produced by any one of methods disclosed herein. Numerous embodiments are further provided that can be applied to any aspect of the present invention described herein.

In one aspect, disclosed herein is a transformed pupae produced by any one of methods disclosed herein. Numerous embodiments are further provided that can be applied to any aspect of the present invention described herein.

In one aspect, disclosed herein is a transformed embryo produced by any one of methods disclosed herein. Numerous embodiments are further provided that can be applied to any aspect of the present invention described herein.

In one aspect, disclosed herein is a transformed larva produced by any one of methods disclosed herein. Numerous embodiments are further provided that can be applied to any aspect of the present invention described herein.

In one aspect, disclosed herein is a population of transformed insects produced by any one of methods disclosed herein, wherein the method further comprising a step of mass rearing the transformed insect, prior to the step of harvesting the biomass. Numerous embodiments are further provided that can be applied to any aspect of the present invention described herein.

### DESCRIPTION OF THE FIGURES

FIG. 1 shows a helper plasmid containing a piggybac transposase under the control of drosophila melanogaster hsp70 promoter.

FIG. 2 shows a donor plasmid containing a cDNA coding for the protein of interest under the control of a functional promoter.

FIG. 3 shows images taken from control (C) and transgenic (T) H. illucens (BSF) 5th instar larvae from our recent expression studies. The mNeonGreen fluorescence in the transgenic (T) larvae is present in clusters, visible across the entire larval body (a portion of the mid-section is shown). No fluorescence was observed in the control (C) larvae.

### DETAILED DESCRIPTION OF THE INVENTION

In describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention. It should be understood that this invention is not limited to the particular methodology, protocols, material, reagents, and substances, etc., described herein. The terminologies used herein are for the purpose of describing particular embodiments only and are not intended to limit the scope of the present invention, which is defined solely by the claims/items.

In the present invention, Hermetia illucens, Gryllodessigillatus, Gryllus assimilis, Acheta domesticus, Tenebrio molitor, Tenebrio obscurus, Alphitobius diaperinus are used as a platform for the production of recombinant proteins of commercial potential. The method for producing a protein of interest, comprises transforming an insect with an expression system containing a recombinant DNA capable of expressing the protein of interest. In the current invention, the host insects are transformed by the gene of interest at different developmental stages ranging from the egg stage , to the pupal stage.

The use of live insects as a production platform for recombinant proteins is a promising approach. In this approach, insects are used as mini-biofactories to obtain the desired protein. Insects are highly efficient protein producers because of their accelerated metabolism When compared to insect cell-lines and other platforms, whole insect-mediated protein expression offers multiple advantages including the ability of insects to produce larger amounts of recombinant proteins coupled with a significant reduction in production costs. Insects as living biofactories constitute a promising alternative to insect cells, conventional fermentation technologies and also to plant-derived proteins because of the production versatility, scalability, automation possibilities, efficiency and speed of development. For example, insects as living biofactories avoid the necessity of bioreactors for the expression of proteins. Bioreactors are a technological and economical barrier to produce new and existing recombinant proteins, since they are inefficient, expensive, technologically complex (taking several years to build, hard to validate, need highly qualified personnel to their manipulation, prone to contamination, and unreliable) In addition, they face the problem of limited scalability. In comparison to insect cell culture systems that produce milligrams/liter of recombinant proteins, the insect larvae can produce up to grams or hundreds of grams/liter of recombinant proteins/liter.

Black Soldier Fly is a non selective insect, feeding on any source of feed while neutralizing pathogenic bacteria which makes the process for protein production much easier. After intensive research, the inventors of the present invention have found a solution to the above problem, namely a protein expression system in eggs, larvae, or pupae belonging to the diptera order, more preferably belonging to the species Hermetia illusion, which is more efficient than other insect species and moreover allows for an unprecedented scale of automation (scale up), which increases the efficiency and reduces costs associated to recombinant protein expression, in particular at industrial scale. Cabbage looper and silkworm have relatively low eggs produced per cycle compared to Black Soldier Fly or mealworm which can produce up to 1000 eggs per cycle. Black Soldier Fly are produced in close proximity with no cocoon formation (unlike Cabbage looper and silkworm) which means they could be produced in small areas. This supports the ability of vertically fanning and stacking them for scale up production.

Technical problems associated with genetic transformation of certain developmental stages in some insect species such as the silkworm are not encountered with the eggs of the Black Soldier Fly. The injection methods developed in other organisms, such as Drosophila and mouse, do not work well in the silkworm as the egg chorion is hard and strong, and the tip of a thin glass capillary cannot penetrate the eggs. However, the eggs of the Black Soldier Fly can be easily penetrated by standard transformation techniques making the establishment of germline transformation a much easier process than the silkworm.

The present invention is thus directed to harnessing the potential of mass-reared insects for scalable recombinant protein production, preferably insects belonging to the species Hermetia illucens. Such insects can grow around 24 times in length and 9,000 times in weight within 18 days from egg hatching.

As used herein in the specification, “one”, “a/an” may mean “at least one” or “one or more,” unless otherwise indicated. As used herein in the claim(s), when used in conjunction with the word “comprising”, the words “a” or “an” may mean one or more than one. As used herein “another” may mean at least a second or more

According to the invention, the term “larva” refers to the immature, wingless, and often wormlike feeding form that hatches from the egg of many insects. It alters chiefly in size while passing through several molts, and finally transforms into a pupa or chrysalis from which the adult emerges.

The term “pupa” refers to a life stage of some insects undergoing transformation. The pupal stage is found only in holometabolous insects, those that undergo a complete metamorphosis, going through four life stages; embryo, larva, pupa and adult.

As used herein, “recombinant DNA” refers to an artificial DNA form that does not naturally exist, which is manipulated through the combination or insertion of one or more DNA strands to combine DNA.

As used herein, “recombinant protein” refers to a protein originating from recombinant DNA. Such proteins may be used for the benefit of humans and animals, and may have industrial, commercial or therapeutic uses.

The recombinant protein of interest produced from the insects biomass could be any protein sequence including but not limited to an antibody, an enzyme, a growth factor, a cytokine, a hormone, a signaling peptide, a structural protein, a transport protein, a storage protein, a fusion protein, an interleukin, an artificially designed protein, a subunit vaccine, monoclonal antibodies, cell surface receptors, hormonal receptors, membrane transport proteins, cyclins, Fab fragments, nanobodies, affibodies and other antibody mimetics, viral antigens, virus-like particles, viral receptors, fluorescent proteins, fusion, cholinesterases, peptidases, kinases, phosphatases, human adenosine deaminase, Phospholipases, invertebrate immune proteins, RAS effectors, antimicrobial peptides or any other known protein

There is no limitation on the origin of the recombinant protein of the invention. Preferably said protein is derived from mammalian, bacterial, viral, fungal, plant or aquaculture proteins.

As used herein, “expression system” includes recombinant DNA elements involved in the expression of a particular gene, such as the gene itself and/or a factor that controls the expression of this gene (e.g., a promoter),

Successful production of transgenic insects can either be achieved by germline or somatic transformation.

As used herein, “transgenic insect” refers to insects that express exogenous nucleic acid either in a transient or stable manner

As used herein, “stable expression” refers the gene expression occurring due to the integration of the exogenous nucleic acid into the genome of the host organism.

As used herein, “transient expression” refers the gene expression occurring due to the presence of an exogenous nucleic acid vehicle inside the cells of the host organism without integrating into the host genome.

As used herein, “Germline transformation” refers to genetic transformation that occurs by targeting the germ cells of the host organism to be inherited and passed on to the next generations.

As used herein, “Somatic transformation” refers to genetic transformation that occurs in the somatic cells of the host organism.

In some embodiments, the genetic transformation is mediated by non-viral expression systems, including but not limited to transposons, recombinases, integrases, ZFNs, TALENs and CRISPR/Cas technologies.

### Transposons

Transposons are genetic elements, which are capable of “jumping” or transposing from one position to another within the genome of a species They are widely distributed amongst animals, including insects. Transposons are active within their host species due to the activity of a transposase protein encoded by the elements themselves, or provided by other means—such as by injection of transposase-encoding mRNA, the use of a second coding sequence encoding transposase or the addition of transposase protein itself. Advances in the understanding of the mechanisms of transposition have resulted in the development of genetic tools based on transposons, which can be used for gene transfer

Any transposable element active in the desired insect may be used. Preferably, however, the transposable element is selected from the group consisting of Minos, mariner, Hermes, Sleeping Beauty and piggyBac.

PiggyBac is a transposon derived from the baculovirus host Trichophisia ni. Its use for germ-line transformation of Medfly has been described by Handler et al., (1998) PNAS (USA) 95:7520-5.

Minos is a transposable element, which is active in Medfly. It is described in U.S. Pat. No. 5,840,865, which is incorporated herein by reference in its entirety The use of Minos to transform insects is described in the foregoing US patent.

Mariner is a transposon originally isolated from Drosophila, but since discovered in several invertebrate and vertebrate species. The use of mariner to transform organisms is described in International patent application WO99/09817.

Hermes is derived from the common housefly. Its use in creating transgenic insects is described in U.S. Pat. No. 5,614,398 incorporated herein by reference in its entirety.

### Site-Specific Recombinases

Site-specific recombinases catalyze the insertion or excision of nucleic acid segments. These enzymes recognize relatively short, unique nucleic acid sequences that serve for both recognition and recombination. Examples include Cre (Sternberg et al, J Mol Biol, 1981, 150:467-486), Flp (Broach, et al, cell 1982, 29:227-234; U.S. Pat. Nos. 5,654,182, 5,885,386; 6,140,129; and 6,175,058) and R (Matsuzaki, et al, J Bacteriology, 1990, 172:610-618). Examples of use of site-specific recombinases for manipulation of nucleic acids are described in U.S. Pat. Nos. 5,527,695; 5,654,182; 5,677,177; 5,801,030; 5,919,676, 6,091,001; 6,110,736; 6,143,557; 6,156,497, 6,171,861; 6,187,994; and 6,262,341. Advances in the understanding of the mechanisms of recombination by these systems have resulted in the development of genetic tools based on them, which can be used for gene transfer.

According to the present invention, Recombinase-mediated cassette exchange (RMCE) technology can be used. RMCE technology allows swapping of large DNA sequences. The technology is based on generating a parental line engineered with a transgene that is flanked by specific recombinase sites (e.g., loxP, lox511). An exchange plasmid containing a second transgene (also flanked by specific recombinase sites) can then be introduced. The exchange plasmid +transgene are transfected into the parental line in the presence of a helper plasmid that expresses recombinase (e.g., Cre). The recombinase catalyzes an exchange between the first transgene integrated in the parental line and the exchange plasmid transgene, thus completing the desired gene transfer

### Zinc Finger Nuclease (ZFNs)

ZFNs belong to a class of artificial restriction enzymes comprising two domains. DNA-binding zinc-finger domain and a DNA-cleavage domain. This class of nucleases could be engineered to target a specific region of the DNA for genomic manipulation. The DNA-binding domains are able to recognize a 9-bp target. The DNA-cleavage domain must dimerize first to be able to induce a double strand break in the target genome (Urnov, F. et al, Nat Rev Genet 2010, 11, 636-646).

### Transcription Activator-Like Effector Nucleases (TALENs)

TALENs are another commonly used gene-editing tool, which similar to ZFNs is comprised of two domains: a DNA-binding domain and a DNA-cleavage domain. The DNA binding domain is derived from Transcription activator-like effectors (TALEs) secreted from bacteria to regulate their respective genes in the host plant cells upon binding specific regulatory elements (Joung, J. K. et al, Molecular cell biology 2013, 14(1), 49-55). TALENs can be engineered to bind specific genomic regions for gene-editing purposes where they introduce a double strand break upon target recognition.

### CRISPR/Cas System

The Clustered Regularly Interspaced Short Palindromic Repeats (CRISPR) and CR1SPR Associated Proteins (Cas) technology has recently emerged as a powerful tool that has revolutionized the field of genome editing owing to its flexibility, high fidelity and design simplicity. CRISPR/Cas system is RNA-guided DNA cleavage system comprising Cas endonuclease enzyme and a guide RNA (gRNA). The gRNA is composed of 2 short RNA molecules bound together known as tracrRNA and crRNA. The trttcrRNA forms the scaffold sequence responsible for binding the Cas endonuclease enzyme, while the crRNA contains the variable sequence (spacer) responsible for binding the target DNA, known as the protospacer. CRISPR/Cas mechanism of action depends on the recognition of a sequence known as protospacer adjacent motif (PAM) by Cas enzyme, followed by the spacer binding to the target region leading to the activation of the Cas endonuclease activity. Cas endonuclease activity results in the formation of a double strand break in the target genomic region, which is then repaired by the cell repair mechanisms to allow for genomic manipulation (Tems M. P., et al, Molecular cell, 2018, 72(3), 404-412).

According to the present invention, the creation of a transformed insect requires that the expression system gets introduced to the host insect by any possible means including but not limited to physical and chemical methods known in the state of the art. In one form of transformation, the DNA is microinjected directly into cells through the use of micropipettes. Alternatively, high velocity ballistics can be used to propel small DNA associated particles into the cell. In another form, the cell is permeabilized by the presence of polyethylene glycol, thus allowing DNA to enter the cell through diffusion. DNA can also be introduced into a cell by fusing protoplasts with other entities, which contain DNA. These entities include minicells, cells, lysosomes or other fusible lipid-surfaced bodies. Electroporation is also an accepted method for introducing DNA into a cell. In this technique, cells are subject to electrical impulses of high field strength which reversibly permeabilizes biomembranes, allowing the entry of exogenous DNA sequences.

The plasmid DNA concentrations used for the genetic transformation include any amount that would result in successful transgenesis. Preferably concentrations used are 10-20, 20-30, 30-40, 40-50, 50-60, 60-70, 70-80, 80-90, 90-100, 100-200, 200-300, 300-400, 400-500, 500-600, 600-700, 700-800, 800-900, 900-1000 ug/ml. Less commonly lower concentrations are used such as 0.1-0.2, 0.2-0.3, 0.3-0.4, 0.4-0.5, 0.5-0.6, 0.6-0.7, 0.7-0.8, 0.8-0.9, 0.9-1 ug/ml

In other embodiments, the genetic transformation is mediated by viral vectors including but not limited to baculovirus, densovirus, FHV virus, vesicular stomatitis virus (VSV) ,lentivirus, ,adenovirus, adeno-associated virus, poxvirus vectors, Epstein-Barr virus, retrovirus, parvovirus, or herpes simplex virus.

According to the invention, the “baculovirus expression vector (BEV)” refers to recombinant baculovirus that has been genetically modified to lead the expression of a foreign gene. The BEV is widely used to express the gene in cultured insect cells and insect larvae. Two of the most common isolates used in foreign gene expression are Autographa Californica multiple nuclear polyhedrosis virus (AcMNPV) and Bombyx mori (silkworm) nuclear polyhedrosis virus (BmNPV). The BEVs are introduced to the insects, which are infected and the virus replicates within the insect.

In BEVs, the foreign gene coding sequence is usually placed under the transcriptional control of a viral promoter. For this reason, usually viral factors are required for the transcription of the foreign gene.

According to the present invention, viral vectors including BEVs can be introduced into insects at any developmental stage through different approaches. One method is infecting insect larvae with virus via oral administration, individual injection, aerosol spray, immersion or by any physical or chemical method.

According to the invention, the larvae or pupae may be stressed before infection with BEVs or any viral vector. The term “stressing” refers to the larvae or pupae being put under stress (e.g., heat shock), without resulting in death. After being stressed, the larvae or pupae will recover and express proteins normally. In some embodiments of the invention, the stressing can be achieved by the following means, alone or in combination: keeping larvae or pupae at a low temperature or a temperature higher than the normal growth temperature but lower than the tolerance temperature, starving larvae or pupae, placing larvae or pupae in an environment with reduced atmosphere, irradiating larvae, or pupae with radiation or treating larvae or pupae with a chemical agent. In some embodiments, the larvae or pupae are kept at a low temperature from about 2° C. to about 15° C., preferably, about 3° C. to about 15° C., more preferably, about 4° C. to about 15° C., about 4° C. to about 12° C., about 5° C. to about 15° C., or about 4° C. to about 10° C., most preferably, about 4° C. to about 6° C., about 4° C. to about 8° C. or about 4° C. to about 10° C. In other embodiments, the larvae or pupae are kept at a higher temperature of about 30° C. to 45° C. preferably, about 32° C. to 45° C., about 32° C. to 42° C., about 32° C. to 40° C., about 35° C. to 45° C. or about 35° C. to 40° C. In some embodiments, the larvae or pupae are treated by irradiating with a low dose of UV. In other embodiments, the larvae or pupae are treated by being placed in an environment with a reduced atmosphere. More preferably, the atmosphere is reduced 5% to 50%. In some embodiments, the larvae or pupae are fasted for at least two days. Preferably, the larvae or pupae are fasted for two, three or four days.

The dose of recombinant viral vector used for the genetic transformation include any dose able to achieve successful infection and transgene expression. Preferably, the multiplicity of Infection (MOI) used is 0.001-0.01 MOI, 0.01-0.1 MOI, 0.1-1 MOI, 1-3 MOI, 3-5 MOI, 5-10 MOI, 10 - 15 MOI, 15-20 MOI, 20-30 MOI, 30-40 MOI, 40-50 MOI, 50-60 MOI, 60-70 MOI, 70-80 MOI, 80-90 MOI and 90-100 MOI.

“Multiplicity of infection” as described herein refers to the number of virions that are added per cell during infection.

“Virions” as described herein refers to the complete infective form of a virus outside a host cell, with a core of nucleic acid and a capsid.

The promoters used to drive the expression of the gene of the exogenous nucleic acids could be any promoter functional in the host organisms, wherein the promoters are either inducible or constitutive promoters. Examples of promoters that can be used include but not limited to HSP70, CMV, CAG, PGK, TRE, U6, UAS, T7, Sp6, lac, araBad, trp, or Ptac.

As used herein, “inducible promoter” refers to promoters that are only active under specific circumstances and can be switched from an OFF to an ON state.

As used herein, “constitutive promoter” refers to an unregulated promoter that allows for continual expression of its associated gene.

A preferred inducible promoter is the heat shock protein HSP70 promoter, which is induced by increasing the temperature at which the larvae are cultured, and tetracycline-inducible expression systems (Heinrich et al. PNAS 2000, 97:8229-8232).

In certain embodiments of the present invention, heat-shock may be used to induce the expression of HSP70, which is induced by elevated temperatures at 29, 30, 31, 32, 33, 34, 35, 36, 37, 38, 39, 40, 41, 42° C.

Other suitable promoters may also be used, such as Mtn promoter, Tet-on, Tet-off systems.

Constitutive promoters used include any promoter functional in the host insect species whether derived from the species itself or any other insect species.

The constitutive promoter may be a cytoplasmic actin promoter. The D. melanogaster cytoplasmic actin promoter has been cloned (Act5C) and is highly active in mosquitoes (Huynh et al, J. Mol. Biol. 1999, 288: 13-20). Cytoplasmic actin genes and their promoters may also be isolated from other insects.

Other examples include the polyubiquitin promoter, cytoplasmic tubulin promoter, d. melanogaster copia LTR, Ds47 , OpIE1, OpIE2

Less commonly, other promoters may also be used, such as D.melanogaster adh, tubulin, PGK, CMV, UBC, or CAGG.

In other embodiments, promoters which control secreted polypeptides may be used, optionally together with appropriate signal sequences, to direct secretion of the protein to the haemolymph. For example, the larval serum protein promoter may be employed

According to the present invention, several approaches could be employed to achieve germline transformation. These approaches include but are not limited to microinjection of desired expression system into the early stage embryos of the host insect. In some embodiments, early stage embryos are microinjected with 0-10, 10-20, 30-40, 40-50, 50-60, 60-70, 80-90, 90-100, 100-110, 110-120 minutes of oviposition. In other embodiments, freshly laid embryos could be stored under specific conditions to delay their development and then used later for microinjection

In another aspect, the invention provides a method for producing a subunit vaccine.

As used herein, a “vaccine” may be defined as a biological agent, and may be defined as a biological agent, that provides an acquired immunity to the particular disease.

“Sub-unit vaccine” as used herein is defined as a vaccine including sub viral components that are post-translationally modified and correctly folded to act as immunogens.

In some embodiments, the subunit vaccine is a virus-like particle. The method involves expressing, in the host organism, one or more viral structural proteins capable of self-assembly to form a virus-like particle. Virus-like particles (VLPs) are multiprotein structures that mimic the organization and structure of standard natural viruses, but lack the viral genome and may produce safer and cheaper vaccine candidates.

In some embodiments, the method further involves isolating the virus-like particle, wherein VLPs can be recovered from the cell supernatant and purified using the same procedure used to purify the virus. VLPs can be engineered to increase the range of immune responses. VLPs can also be engineered to distinguish their immune responses from those induced by infection.

In this specification, the term “particle capable of self-assembly” refers to a particle formed by at least one component that spontaneously assembles. The component may be a polypeptide or a non-peptide compound.

The present invention also relates to the use of red fluorescence proteins such as dsREDs as reporters for gene expression in insect larvae, enabling gene expression of the target to be identified by the naked eye.

In some embodiments, Coral red fluorescence fusion proteins expressed in larvae, such as dsRED, can be discerned by the human eye in normal laboratory light in larvae. The color of the expressed coral red fluorescence proteins is red or pink and are bright enough to be seen by naked eyes under direct sunlight, without the use of any prosthetic tools, thereby enabling the insects that are expressing the target fusion protein to be identified with ease and without any tedious molecular analysis.

The methods of the present invention also related to the isolation of the recombinant protein of interest from insect larvae or pupa by any convenient method. Suitable methods include mixing whole insect larvae/pupa and/or cut up insect larvae/pupa with an extraction buffer for downstream processing to isolate the recombinant protein of interest.

Although whole insect larvae and/or cut up larvae may be utilized, the invention is typically carried out with larvae that are cut or otherwise made not whole. Initially, the larvae may be commuted or cracked.

Prior to comminution, the harvested larvae may be washed to free them of any dirt and particulates and decontaminated to ensure that the larvae are free from pathogens or other contaminants, a key step in order to achieve a safe and high-quality end product.

Prior to being cut, the larvae may be frozen. Freezing can help to minimize homogenization of the larvae. The larvae may be chopped, fractured by impacting, such as with a hammer or mallet, milled or otherwise broken into pieces or comminuted, According to other embodiments, the insects are freeze dried.

In some embodiments, the insect-derived recombinant protein, vaccine, antibody, peptide, or chemical is purified via chromatography purification, distillation, evaporation, adsorption, or crystallization

Black soldier fly (BSF) used in the current invention as an example is a dipteran from the Straiomyidae family commonly found in warm and tropical temperate areas of the world (Hoc, B., et al. PLoS One 2019). The high potential of BSF is attributed to many factors such as their voracious appetite, the short life cycle ranging between 6-7 weeks, and their resilient nature which allows them to thrive under unfavorable conditions (Joly, G., et al. IWMI 2019), Besides their ability to consume a wide range of substrates as food waste, kitchen waste, rice straw, animal manure and faecal sludge, BSFL have been reported to inhibit the growth of harmful bacteria and insect pests (Wang, Y., et al. Foods 2017, 6). In addition, the adult flies do not have mouthparts and they have not been reported to transmit diseases, and they do not feed as well which represents a great advantage in their mass production as they don’t require particular care (Rindhe, S., et al. Int. J. Curr. Microbiol. App. Sci 2019, 8, 1329-1342).The insect grows around 24 times in length and 9,000 times in weight within 18 days from egg hatching.

Tropical house cricket (Gryllodes sigillatus) and house crickets (Acheta domesticus used in the current invention belong to the order Orthoptera, family Gryllidae. Many advantages lie in the rearing of tropical house crickets including their resilience in extreme environmental conditions, their great potential for mass rearing and ability to tolerate high population densities In addition, the protein content in the larvae varies between 60% to 70% (Van Huis, A. et al. Wageningen Academic Publishers 2017) . Jamaican field cricket (Gryllus assimilis) exhibits the same features but to a lesser degree and the protein content varies between 50% to 65%.

The current invention includes as well larvae of different mealworm species such as Tenebrio molitor, Tenebrio obscuras and Acheta domesticus commonly known as yellow mealworm, dark mealworm and lesser mealworm, respectively. These species belong to the order Coleoptera, family Tenebrionidae. Similar to crickets, the larvae thrive under extreme conditions, are suitable for mass rearing and have high protein content ranging from 50% to 65%.

The gene of interest may be introduced to the host insect species via viral or non-viral delivery methods.

In some embodiments, this invention could be used in developing vaccines against the SARS-CoV-2 virus which causes the COVID- 19 pandemic the world is currently facing The process for vaccine development goes through two main, steps. The first is the development and approval. The second phase, which is very challenging and cost ineffective with typical methods, is large scale production. The current invention has the potential to produce kilograms of the vaccine in just 4 days with the current infrastructure. Additionally, its cost is lower, making vaccines viable to distribute worldwide.

In some embodiments, the protein of interest could be an antibody, an enzyme, a cytokine, a hormone, a signaling peptide, a structural protein, a transport protein, a storage protein, a fusion protein, an interleukin, or an artificially designed protein

In some embodiments, the promoters used to drive the expression of the gene of interest could be HSP70, CMV, CAG, PGK, TRE, U6, UAS, T7, Sp6, lac, araBad, trp, or Ptac.

In order to facilitate the practice of the present invention, exemplary procedures are described in the following non-limiting example.

### EXAMPLES

The following will describe the invention further in detail with reference to Examples but the invention is not limited to the following examples.

### Example 1: Rearing the Insect

### Hermetia Illucens

The process of rearing BSF starts from the eggs. The egg hatches after several days in a hatchling container filled with high quality food sources. Following hatching, larvae will feed on a food source with 70% moisture content for five days. The 5~day old larvae are then harvested from the containers and reared on organic waste for approximately two weeks till they are transformed into prepupae. Prepupae are then harvested and placed in dark cages for approximately 10 days to allow for pupation to take place. Following pupation, adult flies emerge into love cages where mating occurs. These cages are supplied with a light source, a wet cloth to hydrate the flies, and a medium suitable for egg laying called eggies. Upon mating, the females deposit the eggs in the eggies and the rearing cycle is dosed.

### Tenebrio Molitor

The process of rearing mealworms starts from the eggs. Eggs are laid from 4 to 17 days after copulation. A single female can generate an average of 500 eggs. The embryonic development lasts from 4 to 6 days, which can be accelerated with a slight increase in temperature (25 to 27° C.). Larval period is reared on a diet of 50% oat flakes, 2.5% brewer’s yeast, and 47.5% wheat flour, at 28° C. and 60% R.H, 8L16D. An average mature larva, weighs 0.2 g and measures 25-35 mm long. After this phase, the larva turns into a pupa, a stage that lasts 5 to 6 days and culminates in an adult individual

### Example 2: Somatic Transformation via Piggybac Transposon System in Specific Body Parts

The gene encoding mNeonGreen Protein from the Branchiostoma lanceolaftum has been expressed in the black soldier fly (BSF). Somatic transgenesis was achieved by the use of piggybac transposon system. The Piggybac system used comprised two vectors: a helper plasmid and a donor plasmid.

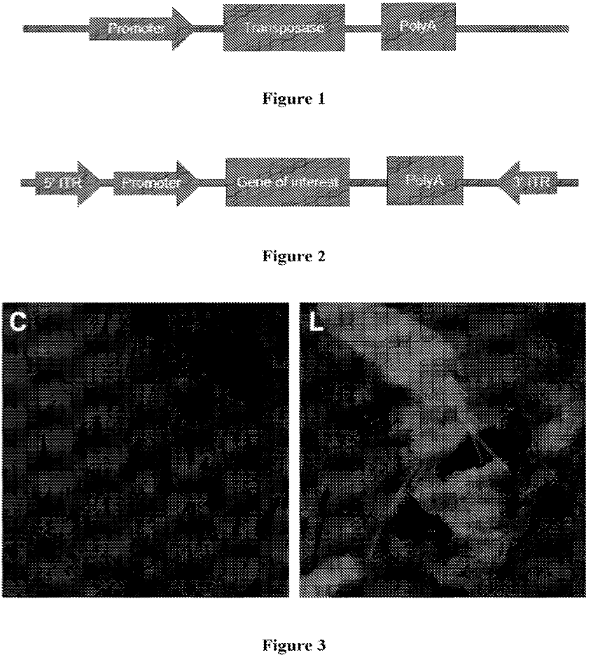

Helper plasmid (FIG. 1) was constructed. It contains a piggybac transposase (PBase) under the control of drosophila melanogasler hsp70 promoter.

Donor plasmid (FIG. 2) was constructed. It contains cDNA coding for mNeonGreen fluorescent protein under the control of drosophila melanogaster hsp70 promoter.

cDNA as described herein is a DNA copy from a messenger RNA

For somatic expression, healthy fifth instar BSF larvae (number of larvae was 55) were injected with 500 nL mixed DNA solution composed of helper and donor plasmid. The concentration of each plasmid was 500 ng/µL after mixing. Immediately after injection an electric shock was applied (15 V). The volume used was 0.25 Helper and 0.25 Donor. The immobilization method used was mechanical between two slides. The larvae were subsequently treated with 37° C. heat shock for 30 min to activate transposase expression.

Expression of mNeonGreen was monitored by detecting mNeonGreen-specific fluorescence in the tissues of larvae after heat-shock treatment at 37° C. for 30 min prior to imaging with fluorescence microscopy. BSF Larvae were screened with a Leica fluorescence microscope using the GFP2 filter set (Leica: exciter filter 480/40 nm, barrier filter 510LP), a filter set compatible with mNeonGreen fluorescence (FIG. 3). 86% of the 55 larvae survived with 80% of the 55 larvae expressing fluorescence.

### Example 3: Germane Transformation of Hermetia Illucens (BSF) via Piggybac Transposon System

### A. By Means of Microinjection

Germline transgenesis is achieved by the use of piggybac transposon system. BSF wild-type strains are used in all experiments; flies are reared under standard conditions. DNA injections of donor plasmids along with a helper plasmid expressing transposase are performed using pre-blastoderm embryos as previously described (Loukeris TG, et al. Science, 1995;270(5244):2002-5), (Rubin GM, et al. Science. 1982 ;218(4570):348-53).

The Piggybac system used comprised two vectors: a. helper plasmid and a donor plasmid. Regarding the donor plasmids, two mNeonGreen constructs are used: one construct uses an inducible promoter (the Drosophila Hsp70) to drive mNeonGreen expression, while the other one used a constitutive promoter (the Drosophila actin5C promoter). BSF Larvae with the Hsp70/mNeonGreen gene show low levels of fluorescence at the normal rearing temperature (22, 23, 24, 25, 26, 27, 28° C.) and elevated fluorescence after exposure heatsock for 10-20 minutes, 20-30 minutes, 30-40 minutes, 40-50 minutes, 50-60 minute. Heatshock temperatures used are 28-29,29-30,30-31,31-32,32-33,33-34,34-35,35-36,36-37, 37-38, 38-39, 39-40, 40-41, 41-42, 42-43, 43-44, 44-45° C. BSF larvae with the actin 5C/mNeonGreen gene show constitutive high levels of mNeonGreen fluorescence. mNeonGreen is expressed in all tissues of BSF. mNeonGreen protein is also detected in transgenic insects using an immunoblot assay.

In a typical transgenesis experiment, a mixture of donor plasmid DNA and helper plasmid DNA are coinjected into pre-blastoderm (0-2 hours post-oviposition) BSF embryos. To detect transgenic BSF, the flies derived from injected embryos (generation G0) are bred by back-crossing to the recipient strain and their progeny are tested individually at the larval stage for expression of either mNeonGreen. Expression of mNeonGreen is monitored by detecting mNeonGreen-specific fluorescence in the tissues of larvae after one or more successive heatshack treatments of larvae (injected by hsp-mNeonGreen plasmid) separated by one day, using standard epifluorescence microscopy.

### B. By Means of Electroporation

Nondechorionated or dechorionated fresh eggs are put into 0.2-cm electrode gap cuvettes (Bio-Rad) with 500-µL electroporation buffer. Various voltages (from 350 V/cm to 1700 V/cm) were used to perform experiments. The dechorionated eggs were fragile, and subject to dessication, Some non-dechorionated eggs were electroporated with 2 pulses separated by a 30-sec interval. Surviving electroporated BSF flies that emerged as adults were individually mated to wild-type insects to initiate the generation of independent fly lines.

Freshly laid BSF eggs were electroporated with 200 ng/µL donor vector containing either EGFP or DsRed and 300 ng/uL helper plasmid coding for hyperactive variant of piggybac transposase in electroporation buffer.

BSF Larvae are screened with a Leica fluorescence microscope using either the GFP2 filter set (Leica: exciter filter 480/40 nm, barrier filter 510LP) or DsRed filter set (Leica: exciter filter 545/30 nm, barrier filter 620/60 nm). Both larvae and adults were used for screening.

Whole frozen larvae expressing DsRed are chopped in a Fitzmill comminuting device, blade side forward, with the sieving screen removed. The larvae are chopped at three different speeds: 3000 rpm, 6000 rpm, and 9000 rpm. The comminuted larvae biomass was mixed with the extraction buffer using end over end mixing. Samples are removed over the course of the extraction for analysis to determine both a rough rate of extraction and to determine the overall level of extraction relative to the whole larvae. The DsRed is detected using either a semi-qualitative SDS-PAGE Coomassie stained gel or with a western blot with a DsRed specific polyclonal antibody, Densitometry on the bands is performed using the Kodak 1 imaging system.

### Example 4: Germline Transformation of T.Molitor via Piggybac Transposon System

T. molitor embryos are collected from overnight oviposition (age up to 24 hr), washed with 2.5% bleach, and positioned on the edge of glass cover slides for microinjection. Donor and helper plasmids are injected at ratio 400ug/ml:100ug/m1 After injections, embryos are incubated in a humidity chamber, and pupae are placed in a Petri dish with T. molitor diet, both at 28° C. and 60% R.H. Larvae emerging from eggs are placed on T. molitor diet and are screened for transgene expression. Pupae are allowed to eclose, and adults were screened for transgene expression.

### Example 5 Baculovirus-Mediated Delivery and Expression of the Transgene

### 1. Production of Baculovirus Expression Vectors

Once the exogenous gene coding for the protein of interest is assembled, it is packaged into a Baculovirus transfer vector. The transfer vector is a plasmid-based vector that contains regions of the viral genome necessary for the integration of the gene of interest into the desired site of the viral DNA. Following the construction of the transfer vector, it is co-transfected along with the viral genome into cultured insect cell-lines for production and amplification of recombinant baculovirus. Recombinant baculoviruses are then isolated and purified from the cultured cells and then delivered into the host insect species at the required titer.

### 2. Delivery of the Recombinant Baculovirus

### A. Injection of the Recombinant Baculovirus Into the Larvae

Recombinant baculovirus containing the gene of interest is constructed under the influence of a strong promoter capable of driving the gene expression in insect larvae. The budded virus form or the bacmid form of the baculovirus is injected into the hemocoel of the BSF larvae in their 5th instar developmental stage. Prior to injection, the larvae are placed in ice to limit their movement and ease their injection. 5-50 µl recombinant baculovirus with a titer of about 1×10⁷ pfu/ ml is used for injection using a microneedle.

“Bacmid” refers to a plasmid construct which contains the nucleic acid sequence that is sufficient for generating a baculovirus when transfected into a cell

### B. Infection of Insect Larvae With Recombinant Baculovirus by Aerosol Infection

Recombinant baculovirus in the occluded form (ODV) is introduced into the insect larvae in the 5th instar developmental stage by means of aerosol infection. Aerosol spray contained 2 ml of the recombinant baculovirus solution at a concentration of 1×10⁷ pfu/ml.

### C. Infection of Insect Larvae With Recombinant Baculovirus by Oral Inoculation

Larvae in their 5th instar are starved for 24 hours then fed with a diet mixed with recombinant baculovirus in the occluded form (ODV).

### 3. Analysis of Transgene Expression

Following the infection of recombinant baculovirus containing a fluorescent protein (mNeonGreen, EGFP or DsRed) by either of the above mentioned routes, the larvae are reared on a special diet and kept in a humidified chamber at 70% humidity, 23-25° C. and exposed for a 16:8 photoperiod (L:D) cycle. The expression is visually monitored by fluorescence microscopy. After they are inoculated and incubated for 4 days, body fluids of the larvae were collected for Subsequent analysis. The proteins contained in the body fluid are analyzed by SDS PAGE electrophoresis (Sambrook and Russell, 2001, Molecular Cloning, A8.40-A8.55). Western Blot immunoassay using PVDF membrane is further conducted.

### Example 6: CRISPR/Cas-Mediated Stable Integration

In this example, genomic integration of gene of interest (GOI) is achieved via CRISPR/Cas technology. As previously mentioned, CRISPR/Cas technologies offer an attractive alternative method for precise stable genomic integration mediated by gRNA and PAM sequence recognition. One way of using CRISPR for genomic insertion is with the use of a target and a helper plasmid, as described by (Hovemann BT, et al. Gene. 1998 Oct 9;221(1): 1-9), (Bassett A., et. al. Methods. 2014 Sep;69(2): 128-36).

CRISPR target sequences are identified through a three- step process. The first step is creating an off target database based on the BSF reference genome. The database is created by looping over the whole genome searching for the PAM sequence of the Cas12a enzyme, TTTN. The second step is discovering possible CRISPR guide RNA target sequences in a region of interest. Both the Actin 5c gene (NCBI ID: LOCI 19646467) and the 2k base region upstream of the gene are searched for discovery. The rationale behind choosing Actin 5c is because it’s one of the consistently in the top 1% highly expressed genes throughout the different life cycles of BSF. Moreover, the gene is surrounded by a highly accessible region of DNA, a defining feature in successful CRISPR integration. The discovery in the gene region yielded 22 possible Cas12a guide RNA target sequences, while the discovery in the upstream region yielded 166 possible target sequences

The third phase of the target sequence identification is ranking the discovered possible Cas12a targets according to appropriate performance matrices. The primary performance matrix utilized is the minimization of matches with the off-target dataset created in step one. If there is a tie between target sequences, the sequences with the least free energy of folding and the better GC content is chosen. Out of 22 Cas12a target sequences discovered inside the Actin 5c gene, three have zero matches in the BSF off target database. The best guide RNA selected, TTTGGGTTGAGTGGAGCCTCGGTC, has 59% GC content and -3.6 free energy. As for the 166 target sequences discovered in the region upstream, the best sequence selected, TTTCAACGGTCGCCAGGCTAGGGT, has 58% GC content and -3.2 free energy.

Enhanced green fluorescent protein (EGFP) isolated from Aequorea victoria is expressed in BSF through two approaches. The first is an integration inside the Actin 5c gene where the donor template encodes the GOI, without a promoter as the knocked-in gene will rely on the transcription machinery of Actin 5c. In this case the gene will be surrounded by 1 k homology arms. The second approach is an integration in the upstream region of the gene, which requires a donor template with a suitable promoter, the drosophila Actin 5c besides the GOI. The longer insert in the donor template requires longer homology arms of 1.5 k to surround the insert.

The helper plasmid contains a gRNA sequence targeting the site under the U6 promoter and Cas12a under the constitutively active promoter, polyubiquitin. BSF larvae with the actin 5C/EGFP show constitutive high levels of EGFP fluorescence. EGFP is expressed in most, if not all tissues of BSF. EGFP protein is also detected in transgenic insects using assays as described in insects manipulated by transposon-based technologies described above. EGFP genomic integration into the target site was confirmed by PCR and sequencing. The term “Primer” as described herein refers to a nucleic acid strand that serves as a starting point for DNA replication.

### Example 7: Comminution to Facilitate Protein Recovery

Comminution of the larvae is performed to increase the flowability of the larvae by forming them into a granular mixture and to increase the surface area of the larvae, decrease the distance required for diffusion of the protein of interest, increase yield of proteins recovered from the larvae and/or provide other benefits. Typically, the larvae are broken into pieces having dimensions of about 1 mm to about 1 cm. The process may utilize pieces anywhere from whole larvae down to pieces about 50 microns. Favorable results may be obtained with a granular having a rough diameter of about 0.5 mm to about 2.5 mm. An alternative embodiment includes pressing the larvae into flakes with dimensions typically about 0.1 mm to about 5 mm and more typically about 0.5 mm to about 2 mm. According to one particular embodiment, the larvae are chopped into granules on the order of about 2 mm.

### 1. Comminution Through Milling

One technique involves comminution of insect larvae with a milling machine. A particular milling machine that may be used is a Fitzmill milling machine. According to one technique, the machine may be operated such that the blades face in the forward direction resulting in a granular form of the frozen larvae. The mill may be operated at various speeds to result in cutting of the larvae to various degrees. For example, the mill could be operated at about 3000 to about 9000 rpm. Specific embodiments have utilized about 3000, about 6000 or about 9000 rpm. Comminutation may be carried out at reduced temperature. For example, the comminutation may be carried out at a temperature range of about -100° C. to about 30° C. One particular embodiment was carried out at about 4° C.

### 2. Comminution Through Conical Milling

A second technique involves comminutation with a. conical mill. A particular milling machine that may be used is a Quadro Comil. The mill may be operated at various speeds and with various configurations to result in cutting of the larvae to various degrees.

### 3. Comminution Through Impactation

A third technique involves comminutation by impactation. This method strikes the larvae. with the flat side of the blades, a hammer or mallet, under extreme cold conditions.

### 4. Comminution Through Sieving or Screening

A fourth technique includes scaling the material to be sized through a “sieving” device or screen. Either the blade or the flat of the striker may be oriented towards the larvae. The size reduction may be controlled by scraping or dragging the larvae over the screen or sieve.

If the larvae are infected with a baculovirus, the processed larvae biomass is further processed by exposure to gamma ray irradiation. The purpose of this irradiation is to inactivate the baculovirus and also to make the powder sterile. Each lot is tested to prove that baculovirus is no longer active by carrying out a bioassay in insect larvae, to examine whether infection occurs.

The pieces of larvae, whole larvae, whole insects and/or pieces of insects may then be mixed with an extraction buffer. The extraction buffer may include any components in which the target protein(s) are soluble. According to one embodiment, the buffer includes 50 mM Tris having a pH of 4 to 8, 0 to 300 nM NaCl, and 0 to 5 mM beta-mercaptoethanol.

The buffer may be mixed either with the insect or with comminuted insect parts at a variety of ratios. The ratio may depend upon the number of runs that are carried out. For example, if multiple runs are carried out, the ratio may be skewed to more insects. An example of a ratio that may be utilized is 1:3 insects to buffer.

Once the insect/insect pieces and buffer are combined, they may be mixed. The mixing may be carried out in a variety of ways with a number of different apparatuses. For example, the mixed together may be carried out with end over end mixing or with an overhead lightening mixer.

After mixing of the insect pieces, the protein(s) of interest may be extracted from the larvae or larval pieces and the pieces are separated from the buffer, which contains the protein(s) of interest. Extraction and clarification can take place either separately or together. The extraction and the separation may be carried out in a number of different ways. For example, decanting, sieving, screening, low speed centrifugation, counter current extraction, decanter centrifugation, hollow tube or large bore hollow fiber or plate and frame tangential flow filtration, and/or percolation extraction could be utilized. More than one extraction and/or separation step may be carried out. Also, one or more different processes may be utilized in the extraction and separation. Both the extraction and the separation technique(s) utilized may depend at least in part upon the protein(s) involved and the size of the insect pieces, among other factors. The extraction may take about 15 to about 45 minutes. The time range for extraction of the protein or material of interest depends largely on the protein or material of interest and on the buffers used in the extraction process and can range from 15 seconds to up to 4 hours.

Other extraction parameters that may be controlled can include temperature, centrifugation speed and nominal molecular weight cut-off (NMWCO) range for potential tangential flow filtration steps. Typically, extraction is carried out at a temperature range of about 4° C. to about 45° C. According to two particular embodiments, extraction may be carried out at a temperature of about 4° C. or about 20° C. Centrifugation for extraction may be carried out at a speed of about 2,000 × g to about 15,000 × g. The nominal molecular weight cut-off (NMWCO) range for potential tangential flow filtration steps for extraction may be about 100 kDa NMWCO to about 0.22 microns.

Clarification may be carried out at a temperature range of about 4° C. to about 45° C. According to one particular embodiment, clarification was carried out at a temperature of about 4° C. Centrifugation that may be carried out for clarification may be carried out at a speed of about 2,000 × g to 15,000 × g. Additionally, clarification may be carried out with a screen size of about 0.01 mm to about 1 mm. One particular embodiment was carried out with a screen size of about 0.5 mm. The nominal molecular weight cut-off (NMWCO) for potential tangential flow filtration steps for clarification may range from about 100 kDa NMWCO to about 0.22 microns.

The invention may be utilized to isolate any protein product or other desired materials or components from the organism involved. For example, any protein that is or could be expressed by insect larvae, whether native or recombinant, could be isolated.

### Example 8: Purification of Recombinant Proteins

After separating the protein-containing buffer from other insect portions, the protein(s) is/are isolated from the buffer. This is carried out utilizing known methods for isolating proteins. For example, proteins are isolated by any combination of tangential flow filtration, liquid-liquid extraction, column chromatography, precipitation, membrane binding, and/or any other known process.

The isolation process(es) is/are carried out until achieving a desired degree of purity of the protein. Typically, the protein is processed until reaching at least about 85% purity. More typically, the protein is at least about 90% pure. In some cases, the protein is at least about 95% pure. The protein is processed until it has a degree of purity that is typically necessary for the protein to have effectiveness for its end use.

### Purification of SARS-CoV2 Spike Protein

This example describes the optimized purification process of the full-length SARS-CoV2 Spike protein in larvae of the black soldier fly, Hermetia illucens. Three chromatographic steps including two ion exchange steps and one intermediate hydrophobic interaction chromatography step are required to reach a purity greater than 95%.

Briefly, the harvested larvae are collected and frozen at -60° C. until they are ready for recombinant protein, total protein, and protease assays. The frozen larvae are thawed, and homogenized in 50 mM Tris having a pH of 4 to 8, 0 to 300 mM NaCl, and 0 to 5 mM micron filter.

The Spike proteins are extracted from the cellular membranes with a non-ionic detergent and insoluble material removed by centrifugation at 10,000 × g for 30 min. The S proteins oligomers are purified using a process including anion exchange, affinity capture and size exclusion chromatography. During the purification process, the detergent concentration is lowered allowing S trimers to form higher ordered protein-protein micellar nanoparticles, Purified S nanoparticles are passed through a 0.2 micrometer filter and stored -80° C.

The SARS~CoV Spike protein samples are analyzed by SDS-PAGE using 4~12% gradient polyacrylamide gels (Invitrogen), stained with GelCode Blue stain reagent (Pierce, Rockford, IL) and quantified by scanning densitometry using OtieDsc-,iii system (BD Biosciences, Rockvine, MD). The total protein concentration of the purified Spike proteins is determined using the (BCA bicinchoninic acid protein assay. Pierce Biochemicals) and S-protein particle size and sample homogeneity is examined by dynamic light scattering using ZETASizer Nano (Malvern !nstruments, PA) using standard, manufacturer recommended methods.beta-mercaptoethanol. The homogenate is centrifuged at 25,000 × g for 30 min at 4° C.: to remove large debrisAfter centrifugation, the supernatant is also further clarified using a 0.22.

### Yield of Recombinant Protein

The yield of recombinant protein is above 2 mg per larvae. Preferably, the yield of recombinant protein is 30 mg - 35 mg per larvae. The larval weight is around 0.3 gm with 40% dry mass. 45% of the dry mass is protein, recombinant protein can reach 10 - 50% of the total protein yield. By using endogenous promoters from the insect and optimizing the codon, the yield is increased to above 60% (~ 35 mg/ larvae in the case of black soldier fly).

### Example 9: Recovery of Recombinant Proteins From the Hemolymph

Recover the suspensions in which the proteins are harbored by recovering the hemolymph from insects which may readily be done by bleeding the insects. Following bleeding, the hemolymph undergoes an oxidation reaction and turns dark and viscous in color. The oxidation reaction is slowed by keeping the samples refrigerated, and by adding an antioxidant, such as glutathione. The hemocytes are pelleted from the hemolymph via centrifugation. The resulting supernatant is assayed or stored frozen for later use. Suitable protein purification systems including high performance liquid chromatography, affinity binding columns, and other similar techniques known to those of ordinary skill in the art are utilized to readily further isolate the proteins of interest from the supernatant.

### Example 10; Production of VLPs in Insect Larvae

### A. VLPs of FMDV Type O/IND/R2/75

VLPs of FMDV type O/IND/R2/75 are produced in insect larvae of Black soldier fly. RecombinantBEVs coding for such VLPs are infected into the BSF insect larvae according to the method described by Kumer et al, 2016 (Kumar, M., et al , 2016. Virusdisease, 27(1), 84-90). Briefly, 50 µL of recombinant baculovirus is injected into the hemocoel of each larva. The larvae are daily observed for changes in the behaviour, feeding habit and mortality due to baculovirus infection. Preparation of whole larval extract and collection of hemolymph is performed on specified time intervals of 1-8 days post infection (dpi).

Hemolymph (2.5 mL) collected from BSF at 1,2,3,4,5,6,7 and 8 dpi is purified through 20-60 % sucrose gradient (0.5 mL each of 20, 30, 40, 50 and 60 % sucrose) by ultracentrifugation at 120000 × g for 16 h at 4° C. in 5 mL polyallomer tubes. After ultracentrifugation, fractions of 0.5 mL each are collected from bottom to top and tested in S-ELISA. S-ELISA is conducted on hemolymph and whole larval extracts. Briefly, the 96-well ELISA . plate is coated with 50 microlitre (µL) of FMDV anti-146S serum raised in rabbit at 1 in 1000 dilution in carbonate-bicarbonate buffer (pH 9.6) and incubated at 37° C. for 1 h. All reagents were added in 50 µL volume for each well. After incubation, the plate is washed three times with PBST (Phosphate buffered saline (PBS) containing 0.05 % Tween-20). Samples are added in duplicates, with four wells each for positive (viral antigen) and negative (Sf9 cellular antigen) controls. The plate is incubated and washed as described earlier. The anti-146S guinea pig tracing antibody diluted 1:4000 in blocking buffer (PBST + 5% adult donor bovine serum) is then added to each well. The plate is washed after incubation at 37° C. for 1 h and anti-guinea pig IgG conjugated to horse raddish peroxidase) at 1:3000 dilution in blocking buffer is added and incubated. After washing, freshly prepared orthophenylene diamine/hydrogen peroxide substrate is added and incubated at 37° C. for 15 min for colour development. Then the reaction is stopped using 1 M H2SO4. The plate absorbance is read at 492 nm in an ELISA reader with 620 nm reference wavelength. Positive- negative cut off value was calculated as twice mean ± standard deviation of four intermediate blank wells.

The amount of VLPs in terms of recombinant protein content of peak fractions of the hemolymph collected from infected BSF larvae is determined by spectrophotometry. The purified VLPs are quantified by spectrophotometric reading at 260 and 280 nm.

### B. HIV Gag 1 VLPs

The human codon-optimised, myristoylated HIV-1 subtype C gag gene was coupled to different regulatory elements and cloned into the piggyBac plasmid. This plasmid was co-transformed with helper plasmid encoding piggybac transposase enzyme into the pre-blastoderm eggs of BSF.

VLPs were then purified from transgenic BSF larvae or pupa (G1) as described by Lynch et al, 2010 (Lynch AG, et al, 2010, BMC Biotechnology, 10:30).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A transformed insect comprising at least one exogenous gene, wherein the insect is of a genus selected from the group consisting of Hermetia, Gryllodes, Gryllus, Acheta, Tenebrio, and Alphitobius.

2. The transformed insect of claim 1, wherein the exogenous gene is comprised within somatic cells of the insect.

3. The transformed insect of claim 1, wherein the exogenous gene is comprised within the germline of the insect.

4. The transformed insect of claim 3, wherein the exogenous gene is stably heritable.

5. The transformed insect according to claim 1, wherein the insect is a species genus selected from the group consisting of Hermetia illucens, Gryllodes sigillatus, Gryllus assimilis, Acheta domesticus, Tenebrio molitor, Tenebrio obscurus, or Alphitobius diaperinus.

6. (canceled)

7. The transformed insect according to claim 1, wherein the insect is a mealworm selected from the group consisting of Tenebrio molitor, Tenebrio obscurus and Acheta domesticus.

8. The transformed insect of claim 1, wherein the insect is an embryo, larva, pupa or adult.

9. The transformed insect of claim 1, wherein the insect expresses a recombinant protein encoded by the at least one exogenous gene.

10-12. (canceled)

13. The transformed insect of claim 9, wherein the recombinant protein is selected from the group consisting of an antibody, an antigen, an antigen-binding molecule, an enzyme, a hormone, a component of a vaccine, and a virus-like particle.

14. The transformed insect of claim 1, wherein the at least one exogenous gene is integrated into the insect genome by transposon-mediated integration.

15. The transformed insect of claim 1, wherein the at least one exogenous gene is under the control of drosophila melanogaster hsp70 promoter.

16. A reared insect derived from the transformed insect of claim 1, wherein the reared insect comprises the at least one exogenous gene.

17-19. (canceled)

20. A pupa derived from the transformed insect of claim 1.

21. A larva derived from the transformed insect of claim 1.

22. An egg derived from the transformed insect of claim 1.

23. An adult insect derived from the transformed insect of claim 1.

24. A biomass produced from the transformed insect of claim 1.

25-26. (canceled)

27. A method for producing a protein comprising:
a. transforming a first stage of an insect selected from the group consisting of Hermetia illucens, Gryllodes sigillatus, Gryllus assimilis, Acheta domesticus, Tenebrio molitor, Tenebrio obscurus, or Alphitobius diaperinus, wherein the transformation results in the integration of an expression cassette into the genome of the insect and wherein the expression cassette comprises a gene encoding a heterologous protein, wherein the heterologous gene is under the control of a heat shock promoter;
b. heat-shocking the transformed insect at a second stage; and
c. harvesting biomass comprising the protein from the transformed insect.

28-29. (canceled)

30. A method for producing a protein comprising:
a. transforming an insect selected from the group consisting of Hermetia illucens, Gryllodes sigillatus, Gryllus assimilis, Acheta domesticus, Tenebrio molitor, Tenebrio obscurus, or Alphitobius diaperinus, wherein the transformation results in the integration of an expression cassette into the germline of the insect and wherein the expression cassette comprises a gene encoding a heterologous protein,
b. breeding the transformed insect to produce a subsequent generation insect; and
c. harvesting biomass comprising the protein from the subsequent generation insect.

31-55. (canceled)

56. A method for producing transformed insect comprising transforming an insect egg or embryo of Hermetia illucens by providing a transposase enzyme active in the insect, and an expression cassette, wherein the expression cassette comprises a gene encoding a heterologous protein and wherein the expression cassette is flanked at each end by a transposable element, whereby the transformation results in the integration of the expression cassette into the genome of the insect.

57-69. (canceled)
